(19) United States
(12) Patent Application Publication
(10) Pub. No.: US 2009/0215892 A1
(43) Pub. Date: Aug. 27, 2009

(54) Octanol Formulations and Methods of Treatment Using the Same

(76) Inventors: Fatta B. Nahab, Gaithersburg, MD (US); Mark Hallett, Bethesda, MD (US); Jeff Serbin, New York City, NY (US); John A. McLane, Warwick, RI (US)

Correspondence Address: CLARK & ELBING LLP, 101 FEDERAL STREET, BOSTON, MA, 02110, US

(21) Appl. No.: 12/083,141

(22) PCT Filed: Oct. 5, 2006
(86) PCT No.: PCT/US06/38892

§ 371 Date: May 5, 2009

Related U.S. Application Data
(60) Provisional application No. 60/724,635, filed on Oct. 7, 2005.

(51) Int. Cl. A61K 31/045; A61K 31/23

(52) U.S. Cl. 514/552; 514/724

(57) ABSTRACT

The invention features pharmaceutical formulations containing octanol and esters thereof. The pharmaceutical formulations are useful for the treatment of involuntary tremors.

### STATEMENT AS TO FEDERALLY SPONSORED RESEARCH

This invention was sponsored in part by NINDS Intramural Research Funds to Dr. Mark Hallett and Dr. Fatta Nahab at the National Institute of Neurological Disorders and Stroke, a part of the National Institutes of Health. The Government has certain rights to this invention.

### BACKGROUND OF THE INVENTION

The invention relates to pharmaceutical formulations containing octanol and esters thereof for therapeutic use. Such pharmaceutical formulations can be utilized to treat involuntary tremors.

Tremors

Tremors are rhythmic, involuntary muscular contractions characterized by shaking movements that can affect the whole body or particular parts of the body such as the head, hands, fingers, eyelids, vocal cords, trunk, and legs. While most people experience a tremor at some time, usually because of fear or excitement, a number of neurological diseases that destroy nerve tissue cause uncontrollable tremor. These include Parkinson's disease and multiple sclerosis. Other causes include stroke or head injury; Wilson's disease, a hereditary disorder in which toxic levels of copper accumulate in the tissues; mercury poisoning; an over-active thyroid gland; and liver encephalopathy. Tremor can also occur as a side effect of drugs.

Tremors are classified according to how severe it is, how often it occurs, and the type of shaking. The severity of tremors varies greatly, and is largely dependant on the underlying condition causing the tremor. For example, certain conditions or factors such as stress, normal aging, hypoglycemia, or caffeine can only cause minor tremors, whereas more severe tremors can be associated with neurological disorders such as Parkinson's disease, essential tremor, or stroke. Tremors can occur occasionally (sporadic), temporarily (episodic), or can occur at certain intervals (intermittent), and are generally classified as postural, rest, or action tremor.

The main causes of tremors can be disease, hereditary (essential), or drugs. Signs and symptoms depend on tremor type and etiology. The most common tremors and their associated signs and symptoms are:

a) Essential Tremor—This is the most common form of all movement disorders. Essential tremors affect 0.4% of the general population and up to 14% of people 65 years and older. Classical essential tremor is predominantly a postural- or action-type tremor and usually the patient has a positive family history of tremor. Other associated symptoms can include mild gait difficulty and, as a group, patients with essential tremor have an increased incidence of hearing impairment.

b) Physiological Tremor—This tremor is a very-low-amplitude fine tremor (between 6 Hz and 12 Hz) that is barely visible to the naked eye. It is present in every normal individual during maintaining a posture or movement. Neurologic examination results of patients with physiologic tremor are usually normal.

c) Enhanced-Physiologic Tremor—This is a high-frequency, low-amplitude, visible tremor that occurs primarily when a specific posture is maintained. Drugs and toxins induce this form of tremor. The suspected mechanism is mechanical activation at the muscular level. Signs and symptoms of drug toxicity or other side effects can or can not be present.

d) Parkinson's Tremor—This tremor type is a low-frequency rest tremor typically defined as a pill-rolling tremor. In some patients, postural and action tremor can also occur. Parkinson's tremor usually occurs in association with other symptoms, such as micrographia, slowness (bradykinesia), and rigidity.

e) Cerebellar Tremor—This is a low-frequency (less than 4 Hz) intention tremor that usually occurs unilaterally. Common causes are multiple sclerosis, stroke, and cerebellar injury. Signs and symptoms of cerebellar dysfunction can be present, including ataxia, dysmetria, dysdiadokinesia and dysarthria.

f) Holmes' Tremor—Holmes' Tremor or rubral tremor designates a combination of rest, postural, and action tremors due to midbrain lesions in the vicinity of the red nucleus. This type of tremor is irregular and slow frequency (4.5 Hz). Signs of ataxia and weakness can be present. Common causes include cerebrovascular accident and multiple sclerosis, with a possible delay of 2 weeks to 2 years in tremor onset and occurrence of lesions.

g) Drug-induced Tremor—This type of tremor can occur as a side effect of drugs including amphetamines, antidepressants, antipsychotics, caffeine, and lithium, and as a result of withdrawal from alcohol or addictive drugs.

h) Tremor Due to Systemic Disease—This tremor usually occurs when the patient is moving or assumes a specific position. Associated symptoms include asterixis, mental status changes, and other signs of systemic illness. Diseases such as thyrotoxicosis and hepatic failure as well as delirium tremens and drug withdrawal are among the common causes.

i) Psychogenic Tremor—This type of tremor can involve any part of the body, but it most commonly affects the extremities. Usually, tremor onset is sudden and begins with an unusual combination of postural, action, and resting tremors. Psychogenic tremor decreases with distraction and is associated with multiple other psychosomatic complaints.

j) Orthostatic Tremor—This type is a variant of essential tremor. This type of tremor occurs in the legs-immediately on standing and is relieved by sitting down. Orthostatic tremor is usually high frequency (14 Hz to 18 Hz), and no other clinical signs and symptoms are present.

Tremors have the potential to interfere with the daily activities of individuals who suffer from them. Most often, fine motor skills are diminished resulting in difficulties performing everyday tasks such as writing. In addition, tremors can also affect the vocal cords, resulting in a shaky or quivering voice, making communication difficult.

Therapeutic Treatments

Current treatments for individuals suffering from tremors can be ineffective with long-term use or produce unwanted side effects. Beta blockers, such as propranolol (commercially sold as Inderal), nadolol and metaprolol, are normally used to treat high blood pressure, but are often prescribed to treat individuals suffering from tremors. These pharmacological agents block the action of neurotransmitters, particularly compounds related to adrenaline. Possible side effects from these treatments include dizziness, fatigue, nausea, impotence, orthostatic hypotension, depression, confusion and memory loss. These medications also cannot be prescribed to individuals with asthma, diabetes or certain heart problems.

Anticonvulsants, such as primidone (Mysoline), acetazolamide, methazolamide, valproic acid and gabapentin, can be effective in people who don't respond to beta blockers. Similar to beta-blockers, these medications also modulate the function of some neurotransmitters. However, side effects are common, including headaches, sedation, confusion, depression, paresthesias, and gastrointestinal disturbances. In double-blind controlled studies, many of these agents, including gabapentin (Neurontin), have proved to be no more efficacious than placebo. There is considerable variation amongst patients treated with these beta-blockers or anticonvulsants, and these agents can have limited efficacy in reducing the effects of tremors on fine motor manipulations.

Benzodiazepines such as diazepam (Valium), alprazolam (Xanax), chlordiazepoxide, and clonazepam can improve tremor in some patients with essential tremor. However, benefits associated with benzodiazepine therapy in these patients can be due, in part, to its anxiolytic effects and are only safe for short-term use due to severe withdrawal side effects. Side effects include excessive sedation, confusion, and memory loss. A number of other agents previously had been tried but showed inconsistent benefit in the treatment of essential tremor. In small trials, the calcium channel blockers nimodipine and nicardipine have shown some promise; however, mirtazapine (Remeron) has shown no consistent benefits.

Botulinum toxin types A and B have been used for dystonia and spasticity and are now being used as a therapeutic option for selected patients with tremor. Botulinum toxin acts through presynaptic inhibition of acetylcholine release at the neuromuscular junction. Recently, intramuscular injections of botulinum toxin in the hand have been used to reduce tremor by weakening local muscles. When used to treat hand tremors, Botox can sometimes cause weakness in your fingers.

Parkinson's disease tremor, which is believed to be related to low levels of dopamine in certain parts of the brain, usually improves with dopaminergic and anticholinergic medications. The dopaminergic agents carbidopa and levodopa are often prescribed as a combination first line approach for the treatment of parkinsonian tremor. Levodopa is taken orally and is converted to dopamine in the brain, resulting in increased brain dopamine concentrations. Carbidopa is added to the levodopa to prevent the breakdown of levodopa before it crosses into the brain. This combination medicine was approved by the FDA in 1988. However, the effectiveness of these drugs is limited, the side effects can be severe, and the daily cost associated with an effective treatment limiting.

In addition to levodopa, other dopaminergic agents include pramipexole, ropinirole, pergolide, and amantadine. The combination of dopaminergic agents and anticholinergics is effective in tremor-predominant Parkinson's disease. Anticholinergics include trihexyphenidyl, benztropine, and procyclidine. The potential side effects of this combination dopaminergic-anticholinergic therapy include dry mouth, blurry vision, urinary difficulty, confusion, nausea, hallucinations, insomnia, leg edema, and livedo reticularis and the effectiveness of these drugs to control the tremors is reduced over time.

Surgery can be an option for people whose tremors are severely disabling and don't improve with medications. Surgical management includes ablative therapy through stereotactic thalamotomy or chronic thalamic deep brain stimulation. The ventral intermediate nucleus of the thalamus is the best target for both ablative and deep brain stimulation surgeries. Contraindications for surgical management of essential tremor include unstable medical illnesses, swallowing difficulty, and marked cognitive problems.

Stereotactic thalamotomy involves destroying a tiny part of the thalamus to relieve tremor on the opposite side of the body. The majority of people who undergo the operation experience substantial relief from essential tremor, but the operation as usually performed can relieve tremors only on one side of your body. Operation on both sides of the thalamus poses a risk of irreversible speech loss and negative side effects concerning balance and coordination.

Chronic thalamic deep brain stimulation (DBS) involves implanting a device called a thalamic stimulator. A pacemaker-like chest unit transmits electrical pulses through a wire to a lead implanted in the thalamus. The pulses interrupt signals from the thalamus that help cause tremors. The pulse generator can be turned on and off by passing a magnet over the chest unit. This procedure doesn't pose the risks of thalamotomy and can be performed on both sides of your brain. Although these surgical techniques are widely available, they should be used with caution and only after exhausting all possible pharmacologic treatment options.

Additionally, stem cell transplantation, in which fetal tissue is implanted in a patient's brain to replace malfunctioning nerves, is an ongoing source of controversial research but has shown some promise in treating conditions such as Parkinson's disease.

One inexpensive agent that has shown promise in treating symptomatic tremors includes ethanol. Ethanol can reduce tremors by reducing or dampening the synchronized oscillations which cause the tremor. Bain reported that ethanol was an effective treatment in 50% of patients with hereditary essential tremor (Bath et al., Brain 1994, 117: 805-824). However, the effective dose for treatment approaches levels inducing intoxication in patients, limiting its usefulness as a therapeutic agent (Rappaport et al. Life Science 1984, 34: 49-54). In addition, the large doses can result in patients not using the treatment regularly because it interferes with daily activities and can have a social or religious stigma associated with its use (Koller et al., Neurology 1985, 35:1660-2).

Given the limitations of current treatments for tremors, alternative therapeutic agents have been sought. One such alternative therapeutic agent includes the administration of aliphatic alcohols, other than ethanol, to treat tremors. U.S. Pat. No. 4,897,426 to Llinas et al., entitled “Method for blocking calcium channels”, describes the use of aliphatic alcohols (C₂-C₁₀ alkyl alcohols), octanols in particular, to treat essential, severe essential, physiological, rubral and Parkinson's-associated tremors. The patent generally discloses that the aliphatic alcohols can be administered orally, or parenterally in solid or liquid form with or without a carrier or diluent.

Octanol

An alcohol is an organic compound in which a hydroxyl group is bound to a carbon atom. Two opposing-solubility trends in alcohols are: the tendency of the polar hydroxyl group to promote solubility in water, and of the hydrophobic carbon chain to resist it. Methanol, ethanol, and propanol are miscible in water. Butanol, with a four-carbon chain, is moderately soluble in water. Alcohols of five or more carbons (pentanol and higher) are fairly insoluble in water due to the comparably large hydrocarbon chain.

Alcohols, like water, can show either acidic or basic properties at the hydroxyl group. With a pKa of around 16-19 they are generally slightly weaker acids than water. Alcohols can also undergo oxidation to give aldehydes, ketones or carboxylic acids, or they can be dehydrated to alkenes. They can react to form ester compounds, and they can, if activated first, undergo nucleophilic substitution reactions.

Alcohols often have an odor described as ‘biting’ that ‘hangs’ in the nasal passages. Ethanol in the form of alcoholic beverages has been consumed by humans since pre-historic times, for a variety of hygienic, dietary, medicinal, religious, and recreational reasons. Ethanol is known to have a depressing effect that decreases the responses of the central nervous system. While infrequent consumption of ethanol in small quantities can be harmless or even beneficial, larger doses can cause acute respiratory failure or death and with chronic use can cause severe health problems, such as liver and brain damage.

Octanol is a simple alcohol with the formula C₈H₁₈O. The different octanols have a hydroxyl group on either carbon 1, 2, 3, or 4 but have the same molecular weight and same molecular formula. 1-Octanol, also known as caprylic alcohol; heptyl carbinol; or 1-hydroxyoctane, is a clear, colorless, slightly viscous liquid mainly used as a defoaming or wetting agent. It is also used as a solvent for protective coatings, waxes, and oils, and as a raw material for plasticizers. Octanols, such as 1-octanol, 2-octanol, 3-octanol, and 4-octanol are generally used as a food additive and perfuming agent because of their sweet taste and smell. 1-octanol is slightly soluble in water, boils at 194.5° C. and is stable under ordinary conditions of use and storage. Carbon dioxide and carbon monoxide can form when heated to decomposition. The toxicity of 1-octanol is rated as LD5O: 1790 mg/Kg, as tested orally in mice. In a published 1989 animal study (Sinton et al., Pflugers Arch. 414:31 (1989)) it was shown that the various octanols (1-octanol, 2-octanol, 3-octanol, and 4-octanol) intraperitoneally (IP) injected all showed some activity in reduction of reducing the hamaline-induced tremors in rats at effective concentrations ranging from 0.06 to 0.26 mg/kg. In 2000, clinical trials began to evaluate the safety and effectiveness of 1-octanol for treating essential tremor. Trial No. 00-N-0062 was a Phase I clinical trial entitled “1-Octanol to treat essential tremor.” It was a NINDS sponsored clinical trial to test the effects of ingestion of 1 mg/kg on essential tremor in humans. In 2001, a second Phase I clinical trial (No. 01-N-0062) was initiated, entitled “1-Octanol to treat essential tremor.” NINDS also sponsored this trial to evaluate the safety and effectiveness of escalating doses of 1-octanol to treat essential tremor in humans. The results of this trial were also published by Shill et al. (Movement Disorders 2002; 17 (supp; 5): S346) in 2002.

In 2002, Bushara et al. (“Octanol for essential tremor: A double blind, placebo controlled trial,” Movement Disorders 2002; 17 (Suppl. 5): 5350) reported the results of the clinical trial evaluating effects of 1-octanol on twelve patients randomized to receive placebo or 1 mg/kg 1-octanol. The report indicates that 1-octanol reduced peak tremor amplitude versus baseline for up to 2 hours, with no improvement seen in the placebo group.

In 2004, Bushara et al. (“Pilot Trail of 1-octanol in essential tremor,” Neurology 62: 122-124) described the randomized trial of twelve patients given a single oral dose of 1 mg/kg of 1-octanol. The study results indicate a decrease in tremor amplitude frequency in patients treated with 1-octanol. In this study the 1-octanol was formulated with anhydrous lactose as a coated capsule. Shill et al. (“Open-label dose-escalation study of oral 1-octanol in patients with essential tremor,” Neurology 2004 62: 2320-2322) described a dose escalation trial wherein twenty one single oral doses of 1-octanol were given to patients with essential tremor. The study indicated that 1-octanol was tolerated in doses up to 64 mg/kg (in the lactose capsule form), with the only side-effect noted being an unusual taste.

Also, in 2004, a Phase II clinical trial (No. 04-N-0147), entitled “Octanol to treat essential tremor” was initiated. Patients were to be randomly assigned to one of two groups. One group would be given 2 to 4 capsules of 1-octanol 3 times a day for 1 week, followed by a 1-week “washout’ period (no treatment), and then 2 to 4 capsules of placebo 3 times a day for 1 week. Following the same dosage schedule, the second group would be given placebo the first week, followed by the washout period and then 1-octanol treatment. Patients would evaluate their tremor daily according to a tremor scale and are also rate according to an alcohol intoxication scale.

Thus, 1-octanol has been tested in a small group of patients that exhibit essential tremor and the 1-octanol treatment has preliminarily demonstrated beneficial results. However, to date, 1-octanol has only been orally formulated as an anhydrous lactose capsule, which is not amenable to large-scale distribution.

### SUMMARY OF THE INVENTION

The present invention provides novel pharmaceutical formulations, routes of administration and dosing regimens for octanols (e.g., 1-octanol, 2-octanol, 3-octanol, and 4-octanol) and esters thereof. The formulations can stabilize the octanol to allow for long-term-storage of the drug. The formulations can also reduce the unpleasant odors and tastes associated with the previously tested octanol formulations of. The present invention also provides methods of treatment of tremors, including disease-induced, drug-induced and essential, by administration of the novel pharmaceutical formulations of octanol, preferably 1-octanol.

In a first aspect, the invention features a pharmaceutical composition including octanol and a pharmaceutically acceptable excipient, wherein the pharmaceutical composition is formulated for enteric release and the octanol is present in an amount sufficient to treat involuntary tremors when administered to a subject. The enteric formulation can be, for example, an enteric coated capsule or an enteric coated tablet. The enteric coating can include, for example, cellulose, polyvinyl polymers, or polyacrylic polymers, or any other enteric formulation described herein.

The invention also features a capsule including octanol and an oil, wherein the octanol is present in an amount sufficient to treat involuntary tremors when administered to a subject. In certain embodiments the oil is an essential oil, such as almond-bitter oil, anise oil, anise star dark oil, gurjun balsam oil, white gurjun balsam, basil oil, bergamot oil, camphor oil, caraway oil, cassia oil, cananga oil, chamomile oil, cherry oil, cinnamon oil, citronella oil, clove stern oil, clove leaf oil, clove bud oil, cognac oil, coriander oil, cubeb oil, eucalyptus oil, eugenol oil, ginger oil, grapefruit oil, jasmine oil, laurel oil, lavender oil, lemon oil, lime oil, mace oil, mandarin oil, mayonara oil, menthol oil, mint oil, nutmeg oil, orange oil, patchouli oil, peppermint yakima oil, peppermint oil, rose oil, sage oil, sassafras oil, spearmint oil, tangerine oil, thyme oil, violet oil, vetiver oil, or wintergreen oil. Alternatively, the oil can be selected from peanut oil, cottonseed oil, safflower oil, sesame oil, olive oil, corn oil, soybean, or any other oil described herein. In one embodiment the capsule includes 100-300 mg of soybean oil and 10-50 mg of lemon oil. The capsules of the invention can include, for example, 50 mg to 1.0 g of octanol. In one embodiment, a pharmaceutical formulation is provided that contains octanol in a pharmaceutically acceptable diluent that allows for long-term storage of the drug. In one embodiment, the pharmaceutically acceptable diluent can be an oil, such as soybean oil. In other embodiments, the diluent can be sesame oil, soybean oil, mineral oil, lemon oil or mixtures thereof. In a particular embodiment, a pharmaceutical formulation containing octanol and soybean oil is provided. In another embodiment, a pharmaceutical formulation containing octanol and soybean oil and/or lemon oil is provided. In further embodiments, the octanol can be formulated with soybean oil, lemon oil, gelatin, glycerin and/or caramel.

The invention further features a pharmaceutical composition including an ester of an octanol and a pharmaceutically acceptable excipient, wherein the ester is present in an amount sufficient to treat involuntary tremors when administered to a subject. Exemplary esters which can be used in the methods and compositions of the invention include, for example, octyl acetate, octyl propionate, and octyl butyrate. Desirably, the pharmaceutical composition is formulated for oral administration, such as a capsule, tablet, syrup, or elixir, or any other oral formulation described herein.

The invention also features a pharmaceutical composition including octanol and a pharmaceutically acceptable excipient, wherein the pharmaceutical composition is formulated for sustained release and wherein the octanol is present in an amount sufficient to treat involuntary tremors when administered to a subject. The pharmaceutical formulation can be a sustained release capsule or tablet, or any other sustained release formulation described herein.

In yet another aspect the invention features a pharmaceutical composition including an ester of an octanol and a pharmaceutically acceptable excipient, wherein the pharmaceutical composition is formulated for sustained release wherein the ester is present in an amount sufficient to treat involuntary tremors when administered to a subject. The pharmaceutical formulation can be a sustained release capsule or tablet, or any other sustained release formulation described herein.

In another aspect the invention features a method of treating an involuntary tremor in a subject in need thereof by administering to the subject a pharmaceutical composition of the invention in an amount sufficient to treat the involuntary tremor. The pharmaceutical composition can be administered to the subject two or three times daily, or by any other regimen described herein.

The invention further features a method of treating an involuntary tremor in a subject in need thereof by administering to the subject a pharmaceutical composition including two or more octanols selected from 1-octanol, 2-octanol, 3-octanol, and 4-octanol, wherein the composition is administered in an amount sufficient to treat the involuntary tremor.

For example, the octanol formulations can be administered at least once, twice, three, four or five times per day. In an alternate embodiment, the octanol formulations can allow for controlled release of the octanol and administered only one or two times per day. In other embodiments, the octanol formulation can be administered as needed by the patient, for example, to diminish symptoms as necessary. Sustained release formulations can be formulated with, but not limited to, the following: HPMC, biodegradable polymers, and/or enteric coatings. In one embodiment, the formulations can be in the form of microspheres.

In yet another aspect the invention features a kit including (i) a pharmaceutical composition including an ester of octanol and (ii) instructions for administering the pharmaceutical composition to a subject for the treatment of involuntary tremors.

In still another aspect the invention features a kit including (i) a pharmaceutical composition including octanol and formulated for enteric release and (ii) instructions for administering the pharmaceutical composition to a subject for the treatment of involuntary tremors.

In another aspect the invention features a kit including (i) a pharmaceutical composition including octanol formulated for sustained release and (ii) instructions for administering the pharmaceutical composition to a subject for the treatment of involuntary tremors.

In another aspect the invention features a kit including (i) a pharmaceutical composition including an ester of an octanol formulated for sustained release and (ii) instructions for administering the pharmaceutical composition to a subject for the treatment of involuntary tremors.

In certain embodiments of any of the above aspects, the octanol is selected from selected from 1-octanol, 2-octanol, 3-octanol, and 4-octanol, or is a mixture of two or more octanols selected from 1-octanol, 2-octanol, 3-octanol, and 4-octanol.

In one embodiment of any of the above aspects, the octanol or ester of the octanol is substantially pure.

In another embodiment of the present invention, the octanol formulation can be administered orally, as a syrup or flavored drink, as a lingual spray, intranasally and/or subcutaneously. Oral formulations can include capsules, such as gelatin capsules. In a particular embodiment, the 1-octanol/soybean oil formulation can be in the form of a capsule, such as a gelatin capsule.

In other embodiments, the pharmaceutical formulations containing octanol or an ester of the octanol can contain 50-1000 mg of the active ingredient. In another embodiment, the formulation can contain at least about 100, 200, 300, 400, 500, 600, 700, 800, 900, 1000, 15000, 2000, or 5000 mg of active ingredient. The octanol or ester of 1-octanol can be administered at a dose of approximately 1-50 mg/kg. In one embodiment, the octanol formulation can be administered at a dose of approximately 1-3 mg/kg. In other embodiments, the formulations of the invention can be administered at a dose that does not cause unwanted side effects, such as sedation and/or intoxication.

In further embodiments, pharmacokinetic analysis can be conducted to determine levels of 1-octanol in the patient. In one embodiment, blood samples from the patient can be analyzed. In a particular embodiment, plasma samples from the patient can be analyzed. In another embodiment, urine samples from the patient can be analyzed. In another embodiment, fecal samples from the patient can be analyzed. In one embodiment, the analysis can be conducted via gas chromatography mass spectrometry (GCMS). In another embodiment, the analysis can be performed via High Performance Liquid Chromatography (HPLC) and/or mass spectrophotometric (MS) detection methods.

The octanol formulations, routes of administration and dosing regimens of the present invention can be used for the treatment of tremors. In one embodiment, the treatment of essential tremor is provided. In a particular embodiment, the treatment of essential tremor with an oral formulation of 1-octanol and soybean oil is provided. In another embodiment, the treatment of drug-induced and/or disease-induced tremor is provided via the methods and compositions described herein. In one embodiment, the drug-induced tremor can be caused by a drug selected from but not limited to the following: cysclosporine, antidepressants, such as selective serotonin reuptake inhibitors (SSRI's; for example, prozac, paxil); amphetamines, antipsychotics, such as haloperidol, lithium, and metoclopramide; and/or caffeine. In one embodiment, the drug-induced tremor can be caused by withdrawal from drugs such as alcohol and/or opiods. In another embodiment, the drug-induced tremor can be caused by a disease selected from but not limited to the following: Parkinson's disease, Multiple Sclerosis, stroke, head injury, Wilson's disease, mercury poisoning, over-active thyroid gland, and/or liver encephalopathy. In still further embodiments, the compositions and methods of the present invention can be used to treat a tremor selected from the group including, but not limited to, physiological tremor, enhanced physiologic tremor, cerebellar tremor, and/or Holmes' tremor.

In further embodiments of the present invention, the octanol formulations and methods of treatment described herein can be administered in combination or alternation with another drug, such as another drug for the treatment of tremor. In one embodiment, 1-octanol formulations, such as 1-octanol can be used in combination and/or alternation with a drug selected from but not limited to the following: beta blockers, such as propranolol, nadolol and/or metaprolol; anticonvulsants, such as primidone, acetazolamide, methazolamide, valproic acid and/or gabapentin; benzodiazepines, such as diazepam, alprazolam, chlordiazepoxide, and/or clonazepam; and Botulinum toxin type A and/or B.

In other embodiments, the octanol formulations and methods of treatment described herein can be used in combination and/or alternation with drugs used to treat Parkinson's disease. In one embodiment, the octanol formulations described herein can be given in combination and/or alternation with a drug selected from but not limited to the following: dopaminergic agents, such as L-dopa and/or levodopa/carbidopa; dopamine agonists, such as bromocriptine, pramipexole, ropinirole and/or pergolide; COMT inhibitors, such as entacapone and/or tolcapone; monoamine oxigenase (MAO) inhibitors, such as selegiline and/or rasagiline; antiexcitatory agents, such as remacemide; anti-viral agents, such as amantadine; anticholinergics, such as trihexyphenidyl, benztropine, procycidine, and/or biperiden; antipsychotics, such as risperidone, olanzapine, quetiapine, and/or clozapine.

In other embodiments, the octanol formulations and methods of treatment described herein can be used in combination and/or alternation with drugs used to treat multiple sclerosis. In one embodiment, the octanol formulations described herein can be given in combination and/or alternation with a drug selected from but not limited to the following: interferon beta 1a, interferon beta 1b, glatiramer acetate, mitoxantrone, azathiprine, cyclophosphamide, cyclosporine, methotrexate, cladribine, methylprednisolone, prednisone, prednisolone, dexamethasone, acth, corticotrophin, carbaxnazepine, gabapentin, topiramate, zonisamide, phenyloin, desipramine, amitriptyline, imipramine, doxepin, protriptyline, cannabis, pentoxiffylline, and/or hydroxyzine.

By “pharmaceutical composition” is meant a composition containing octanol, or an ester of an octanol, formulated with a pharmaceutically acceptable excipient, and manufactured in compliance with the rules of a governmental regulatory agency as part of a therapeutic regimen that includes instructions for the administration of the composition to a subject having tremors or a condition or disease associated with tremors. Pharmaceutical compositions can be formulated, for example, for intravenous administration (e.g., as a sterile solution free of particulate emboli and in a solvent system suitable for intravenous use), or any other formulation described herein. The pharmaceutical compositions of the invention contain octanol, or an ester of an octanol, in an amount sufficient to reduce the frequency, amplitude, or severity of an involuntary tremor in a subject in a clinically relevant manner, or prevent the onset of involuntary tremors in a subject in a clinically relevant manner when administered to a subject.

As used herein, the terms “formulated for enteric release” and “enteric formulation” refer to pharmaceutical compositions for oral administration able to provide protection from dissolution in the high acid (low pH) environment of the stomach. Enteric formulations can be obtained by, for example, incorporating into the pharmaceutical composition a polymer resistant to dissolution in gastric juices, such as Eudragit L100-55, methacrylates, cellulose acetate phthalate, polyvinyl acetate phthalate, hydroxypropyl methylcellulose phthalate, in particular, Aquateric, Sureteric, and HPMCP-HP-55S. An enteric formulation reduces the percentage of the administered octanol released into the stomach by at least 50%, 60%, 70%, 80%, 90%, 95%, or even 98% in comparison to the formulation of Example 1.

By “sustained release” is meant a pharmaceutical composition that upon administration produces a peak plasma concentration at a time that is at least 2, 3, 4, or 5 times that observed following administration of the formulation of Example 1.

By “an amount sufficient” is meant the amount of octanol or an ester of octanol required to reduce the frequency, amplitude, or severity of an involuntary tremor in a subject in a clinically relevant manner, or prevent the onset of involuntary tremors in a subject in a clinically relevant manner. A sufficient amount of octanol or an ester of octanol used to practice the present invention for therapeutic treatment of tremors may vary depending upon the manner of administration, the age, body weight, and general health of the patient. Typically an amount sufficient of octanol will be at least 50 mg of octanol. For esters of octanol an amount sufficient will be a mass that yields at least 50 mg of octanol upon complete hydrolysis of the ester.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

As used herein, the term “substantially pure” refers to a pharmaceutical composition containing an octanol as an active ingredient, or an ester thereof, that is less than 10%, 5%, or even 1% by mass naturally occurring esters of octanol other than active ingredient and less than 10%, 5%, or even 1% by mass oxidation products of octanol (e.g., to the corresponding aldehyde, ketone, or carboxylic acid).

As used herein, the terms “octanol” and “octanols” refer to compounds having the molecular formula C₈H₁₈O, including 1-octanol, 2-octanol, 3-octanol, and 4-octanol.

As used herein, the term “ester” refers to esters of octanol described by formula I:

C₈H₁₇—O—C(O)—R (I)

In formula I, R is a C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇.

As used herein, the terms “alkyl” and the prefix “alk-” are inclusive of both straight chain and branched chain groups and of cyclic groups, i.e., cycloalkyl. Exemplary cyclic groups include cyclopropyl, cyclobutyl, cyclopentyl, and cyclohexyl groups. The C₁₋₇ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₇ alkyls include, without limitation, methyl; ethyl; n-propyl; isopropyl; cyclopropyl; cyclopropylmethyl; n-butyl; iso-butyl; sec-butyl; tert-butyl; and cyclobutyl.

By “C₂₋₇ alkenyl” is meant a branched or unbranched hydrocarbon group containing one or more double bonds and having from 2 to 7 carbon atoms. A C₂₋₇ alkenyl may optionally include a cyclic ring, in which each ring desirably has from three to six members. The C₂₋₇ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl; allyl; 2-cyclopropyl-1-ethenyl; 1-propenyl; 1-butenyl; 2-butenyl; 3-butenyl; 2-methyl-1-propenyl; and 2-methyl-2-propenyl.

By “C₂₋₇ alkynyl” is meant a branched or unbranched hydrocarbon group containing one or more triple bonds and having from 2 to 7 carbon atoms. The C₂₋₇ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₇ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R) —OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₁₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

Other features and advantages of the invention will be apparent from the following Detailed Description and the claims.

### DETAILED DESCRIPTION

### I. Pharmaceutical Formulations

### Octanol

Octanol is an alcohol with the formula C₈H₁₈O. In certain embodiments, the octanol is 1-octanol, 2-octanol, 3-octanol, or 4-octanol. 1-octanol is also known as caprylic alcohol; heptyl carbinol; or 1-hydroxyoctane. 1-octanol is a clear, colorless, slightly viscous liquid.

### Formulations

In one embodiment, a pharmaceutical formulation containing octanol in a pharmaceutically acceptable diluent that allows for long-term storage of the drug is provided. In one embodiment, the pharmaceutically acceptable diluent can be soybean oil. In other embodiment, the diluent can be sesame oil, soybean oil, mineral oil, lemon oil or mixtures thereof. In a particular embodiment, a pharmaceutical formulation containing octanol and soybean oil is provided. In another embodiment, a pharmaceutical formulation containing octanol and soybean oil and/or lemon oil is provided. In further embodiments, the octanol can be formulated with soybean oil, lemon oil, gelatin, glycerin and/or caramel. In a particular embodiment, the octanol is 1-octanol. Alternately, the octanol can be formulated in a tablet with lactose.

In one embodiment, the diluents can be oils. Oils include, but are not limited to Myverol 18-92, acetylated monoglycerides, Alkamuls 719, Alkamuls 620, Miglyol 812 (caprylic/capric triglyceride), canola oil, caprylic/capric triglyceride, cassia oil, castor oil, castor oil hydrogenated, palm oil-hydrogenated soybean oil, Captex 335 (C8/C10 triglycerides from coconut oil), corn glycerides, corn oil, corn oil PEG-6 esters, cottonseed oil, Captex 200 (C8/C10 diesters of propylene glycol of coconut oil), diacetylated monoglycerides, Sesame oil, Soybean oil hydrogenated, Capmul MCM (C₈/C10 mono-/diglycericles from coconut oil), Benzyl Benzoate, Soybean oil, olive oil, PEG vegetable oil, Vegetable oil, Vegetable oil hydrogenated, peanut oil, mineral oil, or vegetable shortening and mixtures thereof.

Pharmaceutical formulations described herein are suitable for oral, topical (including buccal and sublingual), rectal, nasal, vaginal, or parenteral (including subcutaneous, intramuscular, subcutaneous, intravenous, intradermal, intraocular, intatracheal, intracisternal, intraperitoneal, and epidural) administration.

The compositions can, if desired, be presented in a pack or dispenser device which can contain one or more unit dosage forms containing the active ingredient. The pack can for example comprise metal or, plastic foil, such as a blister pack. The pack or dispenser device can be accompanied by instructions for administration.

Formulations can be used as the active ingredient in combination with one or more pharmaceutically acceptable carrier mediums and/or excipients. As used herein, “pharmaceutically acceptable carrier” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

The flavoring may be one or more flavoring oils. For the purpose of this invention, flavoring oils used herein refer to both entire essential oils and the aroma chemicals malting up the essential oils. Essential oils are predominately volatile materials from botanical sources. The most widely used process for the isolation of essential oils is steam distillation of plant matter, although dry distillation and solvent extraction are also used. Essential oils are generally recognized as safe compositions that can be included in ingested materials. Aroma chemicals refer to chemicals which may be synthetic or natural, derived from essential oils, i.e., derived from plants by distillation, expression, or extraction, and which usually carry the flavor of the plant from which they are derived.

Although the invention is not limited to the specific essential oils listed individually in this specification, a number of important essential oils include: almond-bitter oil, anise oil, anise star dark oil, gurjun balsam oil, white gurjun balsam, basil oil, bergamot oil, camphor oil, caraway oil, cassia oil, cananga oil, chamomile oil, cherry oil, cinnamon oil, citronella oil, clove stern oil, clove leaf oil, clove bud oil, cognac oil, coriander oil, cubeb oil, eucalyptus oil, eugenol oil, ginger oil, grapefruit oil, jasmine oil, laurel oil, lavender oil, lemon oil, lime oil, mace oil, mandarin oil, mayonara oil, menthol oil, mint oil, nutmeg oil, orange oil, patchouli oil, peppermint yakima oil, peppermint oil, rose oil, sage oil, sassafras oil, spearmint oil, tangerine oil, thyme oil, violet oil, vetiver oil, or wintergreen oil.

Aroma chemicals include but are not limited to anethole, carvone, citronellal, and camphor.

The flavoring may be one or more natural essences, for example an essence derived from Coffee, Tea, Chamomile, Cocoa, Ginger, Grape, Hazelnut, or Guava.

The flavoring may be one or more natural extracts. For example, the flavouring may be almond extract, anise extract, caraway extract, cardamom extract, celery seed extract, chocolate extract, cinnamon extract, clove extract, coriander extract, dark cocoa extract, grand marnier extract, lemon extract, lemon lime extract, lime extract, mandarin mint extract, orange blossom extract, orange extract, parsley herb extract, rum extract, tangerine extract, tarragon extract, or vanilla extract bourbon.

The flavoring may be one or more artificial flavorings, or natural flavorings, such as a spicy flavor (e.g., cinnamon, clove, jalapeno pepper, mace, or nutmeg); a nutty flavor (e.g., almond, butter pecan, cashew, coconut, English walnut-black, hazelnut, peanut, pecan, pistachio, walnut, and walnut-black); commonly used pharmaceutical flavorings having long lasting taste profiles and well characterised taste masking properties (e.g., anise, apple, apricot, banana, blackberry, blueberry, brandy, butter, butter rum, butterscotch, caramel, champagne, cherry-black, cherry-maraschino, cherry-red, cherry-wild, cherry apricot, cherry mint, coconut, coffee, cognac, cola, cranberry, cream soda, currant-black, egg nog, fennel, ginger ale, grape, grapefruit, grenadine, hazelnut, lemon, lemon-lime, maple, maple walnut, mint orange, passion fruit, peach, pineapple, plum, prune, raspberry, root beer, rum, rum & coffee, sherry, spearmint, tangerine, tutti frutti, or vanilla custard).

Flavorings such as aroma chemicals, natural essences, essential oils, natural extracts, artificial flavors, natural flavors and pharmaceutical flavors are commonly available, for example, from Blue Pacific Flavours & Fragrances, Inc., (1354 South Marion Court, City of Industry, Calif., USA 91745-2418).

Additionally, the formulations can be combined with pharmaceutically acceptable excipients, and, optionally, sustained-release matrices, such as biodegradable polymers, to form therapeutic formulations. A “pharmaceutically acceptable excipient” includes a non-toxic solid, semi-solid or liquid filler, diluent, encapsulating material or formulation auxiliary of any type.

In one embodiment, administration of the active compound is oral. Oral composition will generally include an inert diluent or edible carrier. Formulations suitable for oral therapeutic administration can be presented as discrete units such as, but not limited to, capsules, gel capsules, tablets, caplets, pills, troches or cachets, each containing a predetermined amount of octanol as a liquid, as a solution or a suspension in an aqueous liquid or a non-aqueous liquid, such as soybean oil; or as an oil-in-water liquid emulsion or a water-in-oil emulsion or as a bolus, etc. Pharmaceutically compatible binding agents, diluents, lubricants, glidants, disintegrants, coloring agents and flavoring agents and/or adjuvant materials can be included as part of the composition.

### Capsules/Tablets

In one embodiment, the pharmaceutical formulation is contained in a capsule, tablet pill or troches, suitable for oral administration. In a particular embodiment, the pharmaceutical formulation is contained in a softgel capsule. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include but are not limited to microcrystalline cellulose, gum tragacanth, glucose solution, acacia mucilage, gelatin solution, molasses, polyinylpyrrolidine, povidone, crospovidones, sucrose and starch paste. Lubricants include but are not limited to talc, starch, magnesium or calcium stearate, Sterotes, lycopodium and stearic acid. Diluents include but are not limited to, for example, lactose, sucrose, starch, kaolin, salt, mannitol and dicalcium phosphate. Glidants include, but are not limited to, colloidal silicon dioxide. Disintegrating agents include but are not limited to crosscarmellose sodium, sodium starch glycolate, alginic acid, Primogel, corn starch, potato starch, bentonitc, methylcellulose, agar and carboxymethylcellulose. Coloring agents include but are not limited to, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include but are not limited to natural flavors extracted from plants such as fruits and synthetic blends of compounds which produce a pleasant sensation, such as, but not limited to peppermint and methyl salicylate. Wetting agents include but are not limited to propylene glycol monostearate, sorbitan monooleate, diethylene glycol monolaurate and polyoxyethylene laural ether. Emetic-coatings include but are not limited to fatty acids, fats, waxes, shellac, ammoniated shellac and cellulose acetate phthalates. Film coatings include but are not limited to hydroxyethylcellulose, sodium carboxymethylcellulose, polyethylene glycol 4000 and cellulose acetate phthalate.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. Encapsulating substances for the preparation of enteric-coated oral formulations include cellulose acetate phthalate, polyvinyl acetate phthalate, hydroxypropyl methylcellulose phthalate and methacrylic acid ester copolymers.

### Syrups/Elixirs

In one embodiment, the pharmaceutical formulation can be a liquid pharmaceutically administrable formulation, such as a syrup, elixir or drink. In another embodiment, the pharmaceutical formulation can be contained in a syrup, elixir or drink, suitable for oral administration. Liquid pharmaceutically administrable formulations can, for example, be prepared by dissolving, dispersing, or otherwise mixing an active compound as defined above and optional pharmaceutical adjuvants in a carrier, such as a solid or liquid filler, diluents, to thereby form a syrup, elixir, solution or suspension. A syrup can contain, in addition to the active compounds, sucrose as a sweetening agent and certain preservatives, dyes and colorings and flavors.

Such liquid preparations can be prepared by conventional means with pharmaceutically acceptable additives such as suspending agents, such as sorbitol syrup, cellulose derivatives or hydrogenated edible fats; emulsifying agents such as lecithin or acacia; non-aqueous vehicles such as soybean oil, almond oil, oily esters, ethyl alcohol or fractionated vegetable oils; preservatives such as methyl or propyl-p-hydroxybenzoates or sorbic acid, buffer salts, flavoring agents, coloring agents and sweetening agents as appropriate. Methods of preparing such dosage forms are known, or will be apparent, to those skilled in this art; for example, see Remington's Pharmaceutical Sciences, Mack Publishing Company, Easton, Pa., 15th Edition, 1975.

In one embodiment, the pharmaceutical formulation can be suitable for parenteral administration. The pharmaceutical formulation can also be mixed with other active materials, suitable for parenteral administrations, that do not impair the desired action, or with materials that supplement the desired action. Formulations suitable for used for parenteral, intradermal, subcutaneous, or topical application can include the following components: a sterile diluent such as water for injection, saline solution, aqueous and non-aqueous sterile suspensions which can include suspending agents and thickening agents, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. If administered intravenously, preferred carriers are physiological saline or phosphate buffered saline (PBS). The formulations can be presented in unit-dose or multi-dose containers, for example, sealed ampoules, disposable syringes and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules, and tablets of the kind previously described above.

Pharmaceutical organic or inorganic solid or liquid carrier media suitable for enteral or parenteral administration can be used to fabricate the formulations. Gelatin, lactose, starch, magnesium stearate, talc, vegetable and animal fats and oils, gum, polyalkylene glycol, water, or other known carriers can all be suitable as carrier media.

### Buccal/Lingual Spray

In another embodiment, the pharmaceutical formulations provided can be suitable for buccal or sublingual administration. Formulations suitable for buccal or sublingual administration include for example, tablets or lozenges, having the ingredients in a flavored basis, usually sucrose and acacia or tragacanth; pastilles, having one or more of the formulations disclosed herein in an inert basis such as gelatin and glycerin, or sucrose and acacia and lingual sprays and mouthwashes, having one or more of the formulations disclosed herein administered in a suitable liquid carrier. The components of aerosol formulations include solubilized active ingredients, antioxidants, solvent blends and propellants for solution formulations, and micronized and suspended active ingredients, dispersing agents and propellants for suspension formulations.

### Nasal

In other embodiments, the pharmaceutical formulations provided can be suitable for nasal or intranasal administration. Formulations suitable for nasal administration, when the carrier is a solid, include a coarse powder having a particle size, for example, in the range of approximately 20 to 500 microns which is administered by rapid inhalation through the nasal passage. When the carrier is a liquid, for example, a nasal spray or as nasal drops, one or more of the formulations can be admixed in an aqueous or oily solution, and inhaled or sprayed into the nasal passage. For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount, Capsules and cartridges of, for example, gelatin for use in an inhaler or insufflator can be formulated containing a powder mix of the compound and a suitable powder base such as lactose or starch.

### Topical

In one embodiment, the pharmaceutical formulations provided are suitable for topical administration. Formulations suitable for topical administration to the skin can be presented as ointments, creams, gels, and pastes, having one or more of the formulations administered in a pharmaceutical acceptable carrier. The formulations can contain additional agents, such as cleaning agents, wetting agents, sequestering agents, tonicity agents, nutrient agents, contact lens conditioning agents, antioxidants, pH adjustors, and the like. These additional components can be included in the present formulations in an amount effective to impart or provide the beneficial or desired property to the formulations.

### Rectal

In another embodiment, the pharmaceutical formulations provided are suitable for rectal administration. Formulations for rectal administration can be presented as a suppository with a suitable base containing, for example, cocoa butter or a salicylate.

### Vaginal

In other embodiments, the pharmaceutical formulations provided are suitable for vaginal administration. Formulations suitable for vaginal administration can be presented as pessaries, tampons, creams, gels, pastes, foams or spray formulations containing one or more of the formulations and appropriate carriers.

### Controlled or Sustained Release

In one embodiment, provided is a pharmaceutical formulation having a controlled release, delayed release, or combined delayed and controlled release profile. Alternatively, the formulation can present a combination of an immediate release formulation and a controlled release formulation.

The controlled-release of octanol can be controlled in any way suitable for achieving the desired result. Books describing methods of controlled delivery that are appropriate for the delivery of octanol include: Robert S. Langer, Donald L. Wise, editors; Medical applications of controlled release (Volumes 1 and 2); Boca Raton, Fla.: CRC Press, 1984; and William J. M. Hrushesky, Robert Langer and Felix Theeuwes, editors; Temporal control of drug delivery (series); New York: New York Academy of Sciences, 1991. Representative, non-limiting systems encompassed by the present invention include diffusion-controlled, solvent-controlled and chemically-controlled systems.

Due to their polar chemical structure, octanols are often able to solubilize or partially dissolve other chemicals and may solubilize the chemical or ingredients used in encapsulation and tablets; and it has been observed that 1-octanol in a standard gelatin based soft capsule partially weakened the capsule and likely changed potential dissolution. Therefore; the following embodiments include selection of the capsule or tablet ingredient based on being able to maintain structural and appropriate dissolution integrity and not alter substantially the characteristic dissolution pattern of the selected formulation by in-vitro dissolution testing specified by regulatory agents for pharmaceutical uses. The following embodiments contain descriptions of chemicals that may possess these properties.

In one embodiment, controlled-release of octanol is achieved using a diffusion-controlled system, which can be for example, a reservoir device, such as a membranes, capsules, microcapsules, liposomes, or hollow fiber; or a monolithic (matrix) device, such as a polymer matrix. In a particular embodiment of the invention, a polymeric film coating is an enteric polymeric film coating allows the coated solid to pass intact through the stomach to the small intestine, where the drug can then be released for absorption through the intestinal mucosa into the human body where it can exert its pharmacologic effects. Non-limiting examples of enteric polymers include cellulose, vinyl, and acrylic derivatives. In another embodiment, sustained release of octanol can be achieved through microencapsulation. The microencapsulation drug delivery system of the present invention can utilize a variety of protective wall or covering materials, including without limitation, proteins, polysaccharides, starches, waxes, fats, polymers and resins. Polymers can be natural, synthetic or synthetically modified natural polymers. Representative, non-limiting polymers include gelatins, fish collagens, rubber arrabicum, silicon rubber albumen, fibrinogens, casein, haemoglobin, zein, alginate, nylon, nylon-polyethylenimine carragheen, agar-agar, chitosan, arabino-galactan, gelan, cellulose, polyvinylalcohol, polyacroleins, polylactic acid, polyglycolic acid polyamides, polyethyleneglycols, ethyl Styrolmaleinacidanhydride copolymers, cellulose sulphate-poly(dimethyldiallyl)-ammonium chloride, hydroxy-ethyl methacrylate-methyl methacrylate, chitosan-carboxymethyl-cellulose and alginate-polylysine-alginate. In a particular embodiment, the microcapsulate of the present invention utilizes a substantially water-insoluble polymer.

Polymers suitable for use in the formation of monolithic matrix devices include naturally occurring polymers, synthetic polymers and synthetically modified natural polymers. The monolithic matrix device of the present device can also contain polymer derivatives. As used herein, “derivatives” include polymers having substitutions, additions of chemical groups, for example, alkyl, alkylene, hydroxylations, oxidations, and other modifications routinely made by those skilled in the art.

Any polymeric plastic material is suitable for use in the present invention provided it is insoluble or substantially insoluble in water, and includes cellulose derivatives such as cellulose acetates, (cellulose acetate butyrate, cellulose acetate propionate, cellulose acetate phthalate, etc.), methyl, ethyl and propyl celluloses; polycarbonates; polystyrenes; alkylacrylates such as polymethyl methacrylate, polyethyl ethacrylate, polyethylene, polyethylene methacrylate and other lower alkyl acrylates; vinyl acetate/vinyl chloride, methyl acrylate/methylmethacrylate vinyl polymers; polyvinylchloride polyurethanes; polyacrylonitriles; and mixtures, combinations and multipolymers (copolymers, terpolymers, etc.) thereof.

Solvent activated systems include (i) swellable controlled-release systems, such as a hydrogel; (ii) osmotic systems (i.e., involving transport of water through a semipermeable membrane).

In one embodiment, the polymer matrix can be a hydroxypropylmethylcellulose (HPMC) matrix. (See generally Hogan J E. “Hydroxypropylmethylcellulose sustained release technology, Drug Dev. Ind. Pharm. (1989) 15, 975-999). The HPMC matrix can include an HPMC homopolymer, co-polymer or terpolymer. A single HPMC or a mixture of HPMCs of difference molecular weight and structure can be used. HPMC can be used alone, or in a combination with a second polymer type to form a polymer blend.

Osmotically controlled systems are also suitable for use in the present invention. In this embodiment, an osmotic pressure gradient is created to draw an aqueous fluid into a compartment containing octanol, causing octanol to be delivered. Osmotic delivery systems include a compartment containing octanol and an osmotic agent which thaws an aqueous fluid through the walls of the compartment, causing swelling of the osmotic agent and delivery of octanol.

In one embodiment, the drug delivery system is a chemically controlled system. Chemical control can be achieved, for example, using bioerodible polymers or pendant chains.

In one embodiment, controlled release of octanol is achieved using a biodegradable monolithic polymer matrix. In this type of system, the bioactive agent is ideally distributed uniformly throughout a polymer in the same way as in monolithic systems.

Biologically degradable polymers are polymers which degrade to smaller fragments due to chemicals present inside the body. In generally, biologically degradable polymers are either (i) biodegradable polymers or (ii) bioabsorbable polymers. Biodegradable polymers degrade to smaller fragments by enzymes, whereas bioabsorbable polymers degrade in the presence of other chemicals in the body.

Biodegradable polymers include (i) naturally occurring polymers; (ii) modified natural polymers (i.e., chemically or enzymatically modified polymers; and (iii) synthetic polymers. Representative, non-limiting, naturally occurring biodegradable polymers include alginate, dextrin, cellulose, collagen, chitosan and proteins such as albumin, zein and copolymers and blends thereof, alone or in combination with synthetic polymers. In general, these materials degrade either by enzymatic hydrolysis or exposure to water in vivo, by surface or bulk erosion.

In another embodiment, geometrical-physical systems are used to provide controlled-release octanol. This type of system incorporates octanol into a layer a layer or core, which is then formed into a pellet and altered by physical means to effect and control the rate or erosion or dissolution of the dosage form. Surface-area modifications are used to retard the burst release or increase the extent of the release of octanol from tablet cores that possess diffusion limitations. The physically-altered pellet can then be incorporated alone or in combination with other modified pellets and excipients into a capsule or tablet. Representative geometrical-physical systems include enteric-coated tablet, modified-core tablet systems (e.g., Procise®, GlaxoSmithKline; Smartrix®, Smartrix Technologies).

Non-limiting examples of other polymers suitable for use in the controlled-release drug delivery system according to the present invention include gelatins, fish collagens, rubber arrabicum, silicon rubber albumen, fibrinogens, casein, haemoglobin, zein, alginate, nylon, nylon-polyethylenimine carragheen, agar-agar, chitosan, arabino-galactan, gelan, cellulose, polyvinylalcohol, polyacroleins, polylactic acid, polyglycolic acid polyamides, polyethyleneglycols, ethyl Styrolmaleinacidanhydride copolymers, cellulose sulphate-poly(dimethyldiallyl)-ammonium chloride, hydroxy-ethyl methacrylate-methyl methacrylate, chitosan-carboxymethyl-cellulose, alginate-polylysine-alginate, cellulose ester, cellulose ether, an acrylic polymer, ethyl cellulose, cellulose acetate, cellulose acetate butyrate, poly(lactide-co-glycolide) (PLGA), poly(lactic acid) (PLA), poly(glycolic acid) (PGA), polyvinyl chloride, polyethylene, vinyl acetate/vinyl chloride copolymers, polymethylmethacrylates, polyamides, silicones, polystyrene low density polyethylene, ethylene-vinylacetate copolymers, styrene-butadiene-styrene copolymers, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), hydro glycerides (e.g., mono-, di- or triglycerides such as stearin, palnitin, laurin, myristin, hydrogenated castor or cottonseed oils, precirol), fatty acids and alcohols (e.g., stearic, palmitic or lauric acids; stearyl, cetyl or cetostearyl alcohols), fatty acid esters (e.g., monostearates of propylene glycol and of sucrose, sucrose distearate), waxes (e.g., white wax, cachalot wax), hydrogenated castor oil (HCO), ethylcellulose, poly(hydroxy acids), poly(lactic acid), poly(glycolic acid), poly(lactic acid-co-glycolic acid), poly(lactide), poly(glycolide), poly(lactide-co-glycolide), polyanhydrides, polyorthoesters, polyamides, polycarbonates, polyalkylenes, polyethylene and polypropylene, polyalkylene glycols, poly(ethylene glycol), polyalkylene oxides, poly(ethylene oxide), polyalkylene terepthalates, poly(ethylene terephthalate), polyvinyl alcohols, polyvinyl ethers, polyvinyl esters, poly(dimethyl silicone) polymethacrylate, polymethylmethacrylate, polyvinyl halides, poly(vinyl chloride), polyvinylpyrrolidone, polysiloxanes, poly(vinyl alcohols), poly(vinyl acetate), poly(ethylene/vinyl acetate)polystyrene, polyurethanes, derivativized celluloses, alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, polymers of acrylic acid, methacrylic acid, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(butyric acid), poly(valeric acid), poly(lactide-co-caprolactone), polyphosphazenes, poly(vinyl alcohols), polyamides, polycarbonates. polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, polyacrylates, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate), albumin, prolamines, cellulose, dextrans, polyhyaluronic acid, polyhydroxyalkanoates, polyhydroxybutyrate, alkyl celluloses, hydroxyalkyl celluloses, nitrocelluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, ethylcellulose, polyvinyl alcohol, hydroxypropylmethylcellulose, polymethylmethylacrylate, ethyl acrylate, polyethylene, polyvinylacetate, polymethacrylate, styrene/maleic copolymer, cellulose acetate pthalate, cellulose acetate pthalate/PEG blend, microcrystalline cellulose, polydextrose, lactose, shellacs, cellulose derivatives, non-cellulose polysaccharides, polyethylene oxide, polyvinyl alcohols, acrylic acid copolymers methylcellulose, hydroxypropyl methylcellulose (IHPMC) (high, medium and low molecular weight), hydroxyethyl cellulose, hydroxypropyl cellulose, carboxymethylcellulose, hydroxomethylcellulose, hemicellulose, methylcellulose, galactomannans, guar gum, carob gum, gum arabic, sterculia gum, agar, alginates, carbopols 934P and 974P, polyvinyl alcohol (PVA)/polyvinyl pyrrolidone (PVP), gum tragacanth, locust bean gum, karaya gum, proteinaceous substances (e.g., pectin, carrageen) carboxypolymethylene, gelatin, bentonite, magnesium aluminum silicate, carbomer, zooglan, polysaccharides, modified starch derivatives (e.g., Amazio 721A), hydrophilic vinyl acrylic polymers, poly(2-hydroxyethyl methacrylate), poly(acrylic acid), poly(methacrylic acid), poly(N-vinyl-2-pyrohdinone) poly(vinyl alcohol) polyanhydrides, polyesters, polyacrylic acids polyurethanes, pollyphosphoesters and polyphosphazenes and poly(methyl methacrylates, polyanhydrides, poly(ortho)esters, polyurethanes, poly(butyric acid), poly(valeric acid), poly(lactide-co-caprolactone), poly(ethylene terephthalate), poly(butyric acid), poly(valeric acid), poly(lactide-co-caprolactone), polyanhydrides, starch-polyester alloys; styrene-maleic anhydride copolymers, poly(methylvinyl ether-maleic acid), starch, starch-PCL blends, polylactic acid (PLA)-starch blends, polylactic acid, poly(lactic acid-glycolic acid) copolymers, polylactide, polyglycolide; polyactide co-glycolide PC, starch esters, starch ester-aliphatic polyester blends, modified corn starch, polycaprolactone, poly(n-amylmethacrylate), ethyl cellulose, wood rosin, polyvinylalcohol (PVOH), polyhydroxybutyrate-valerate (PHBV), biodegradable aliphatic polyesters, polyhydroxybutyrate (PHB) and polyhydroxy acids.

The controlled-release formulation can also include a number of other excipients and diluents. The term “excipient’ refers to substances that are commonly provided within finished dosage forms, and include vehicles, binders, disintegrants, fillers (diluents), lubricants, glidants (flow enhancers), compression aids, colors, flavors sweeteners, preservatives, suspending/dispersing agents, film formers/coatings and printing inks.

Lubricants can include, for example, magnesium stearate, calcium stearate, zinc stearate, powdered stearic acid, hydrogenated vegetable oils, talc, polyethylene glycol, and mineral oil;

Disintegrants include starches such as corn starch, potato starch, pregelatinized and modified starches thereof cellulosic agents such as Ac-di-sol, montmorrilonite clays, cross-linked PVP, sweeteners, bentonite and VEEGUM™, microcrystalline cellulose, alginates, sodium starch glycolate, gums such as agar, guar, locust bean, karaya, pectin and tragacanth. The present formulations can also contain flavorants or sweetening agents.

The octanol-containing pharmaceutical formulation can require particular binders in order to obtain a suitable control-release product. Suitable binders include but are not limited to cellulose acetate butyrate, cellulose acetate propionate, cellulose propionate high molecular weight (200,000), cellulose propionate medium molecular weight (75,000), cellulose propionate low molecular weight (25,000), cellulose acetate, cellulose nitrate, ethylcellulose, polyvinyl acetate, polyvinylpyrrolidone, vinyl alcohol polymer, polyethylene oxide, water soluble or water swellable cellulose and starch derivatives, acacia, tragacanth, gelatin, starch, cellulose materials such as methyl cellulose and sodium carboxymethyl cellulose, alginic acids and salts thereof, polyethylene glycol, guar gum, polysaccharide, sugars (e.g., lactose, sucrose), invert sugars, poloxomers (PLURONIC™ F68, PLURONIC™ F127), collagen, albumin, gelatin, cellulosics in nonaqueous solvents, pregelatinized starch, starch paste and combinations of the above and the like. Other binders include, for example, polypropylene glycol, polyoxyethylene-polypropylene copolymer, polyethylene ester, polyethylene glycol, polyethylene sorbitan ester, polyethylene oxide or combinations thereof and others known to those of ordinary skill in the art.

The pharmaceutical formulations can also contain diluents. The term diluent is intended to mean inert substances used as fillers to create the desired bulk, flow properties, and compression characteristics in the preparation of tablets and capsules. Such compounds include, by way of example and without limitation, dibasic calcium phosphate, kaolin clay, fructose, sucrose, dextrose, lactose, mannitol, microcrystalline cellulose, powdered cellulose, precipitated calcium carbonate, sorbitol, calcium sulfate, starch and the like. The formulations can contain colorants.

The differences in water solubility of the different octanols may provide a means to increase the concentration of the octanol per weight of tablet, capsule or other oral formulations.

| | Solubility in Water |
|---|---|
| Octanol isomer | at 40° C. (Wt %) |
| 1-octanol | 0.065 |
| 2 octanol | 0.089 |
| 3 octanol | 0.108 |
| 4-octanol | 0.095 |

A higher water solubility would allow a higher loading of the octanol per mg of final formulation

### II. Dosing Regimens

In other embodiments, the pharmaceutical formulations containing octanol can contain 100-1000 mg of octanol. In another embodiment, the formulation can contain at least about 100, 200, 300, 400, 500, 600, 700, 800, 900, 1000, 1500, 2000, or 5000 mg of octanol. In a particular embodiment, 800 mg of octanol can be formulated in a gelatin capsule with soybean oil.

The dose of the compound for the treatment of tremors can also be in the range from about 1 to 50 mg/kg, preferably 1 to 20 mg/kg, of body weight per day, more generally 0.1 to about 100 mg per kilogram body weight of the recipient per day. Lower doses can be, for example, doses of 0.5-100 mg, 0.5-50 mg, 0.5-10 mg, or 0.5-5 mg per kilogram body weight per day.

The compound is conveniently administered in unit any suitable dosage form, including but not limited to one containing 7 to 3000 mg, preferably 70 to 1400 mg of active ingredient per unit dosage form. An oral dosage of 50-1000 mg is usually convenient, including in one or multiple dosage forms of 50, 100, 200, 250, 300, 400, 500, 600, 700, 800, 900 or 1000 mgs. Lower doses can be preferable, for example from 10-100 or 1-50 mg. Also contemplated are doses of 0.1-50 mg, or 0.1-20 mg or 0.1-10.0 mg. Furthermore, lower doses can be utilized in the case of administration by a non-oral route, as, for example, by injection or inhalation.

The concentration of active compound in the drug composition can depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time. As used herein, pharmaceutical composition means therapeutically effective amounts of the agent together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, poiglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally. The dosage can be in the range of approximately 5-80 mg/day. In another embodiment the dosage is in the range of approximately 35-66 mg/thy. In another embodiment the dosage is in the range of approximately 40-60 mg/day. In another embodiment the dosage is in a range of approximately 45-60 mg/day. In another embodiment the dosage is in the range of approximately 15-25 mg/day. In another embodiment the dosage is in the range of approximately 55-65 mg/day. In another embodiment the dosage is in a range of approximately 45-60 mg/day. The dosage can be approximately 60 mg/day. The dosage can be approximately 20 mg/day. The dosage can be approximately 45 mg/day.

Further, as used herein pharmaceutically acceptable carrier are well known to those skilled in the art and include, but are not limited to, 0.01-0.1M and preferably 0.05M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives can also be present, such as, for example, antimicrobials, antioxidants, collating agents, inert gases and the like.

It will be understood, however, that the total daily usage of the formulations can be decided by the attending physician within the scope of sound medical judgment. The specific therapeutically effective dose level for any particular host can depend upon a variety of factors, including for example, the disorder being treated and the severity of the disorder; activity of the specific formulation employed; the specific formulation employed, the age, body weight, general health, sex and diet of the patient; the time of administration; route of administration; rate of excretion of the specific compound employed; the duration of the treatment; drugs used in combination or coincidental with the specific formulation employed; and like factors well known in the medical arts. In addition, the protocols and methods of treatment can be as described previously, for example, as described in Clinical Trial No. 00-N-0062 entitled “1-Octanol to treat essential tremor”; Clinical Trial No. 01-N-0062 entitled “1-Octanol to treat essential tremor”; Shill et al. (Movement Disorders 2002; 17 (supp; 5): S346); Bushara et al. (“Octanol for essential tremor: A double blind, placebo controlled trial,” Movement Disorders 2002; 17 (Suppl. 5):S350); Bushara et al. (“Pilot Trail of 1-octanol in essential tremor,” Neurology 62: 122-124); Shill et al. (“Open-label dose-escalation study of oral 1-octanol in patients with essential tremor,” Neurology 2004 62: 2320-2322); and Clinical Trial No. 04-N-0147, entitled “Octanol to treat essential tremor”, which are incorporated herein by reference.

### Pharmacokinetic Analysis

In one embodiment, pharmacokinetic analysis can be conducted to determine levels of octanol in the patient. In one embodiment, blood samples from the patient can be analyzed. In a particular embodiment, plasma samples from the patient can be analyzed. In another embodiment, urine samples from the patient can be analyzed. In another embodiment, fecal samples from the patient can be analyzed. In one embodiment, the analysis can be conducted via gas chromatography mass spectrometry (GCMS). In one embodiment, an Agilent G1888 Network Headspace Sampler dual-column gas chromatography system can be used. In another embodiment, the analysis can be performed via High Performance Liquid Chromatography (HIPLC) and mass spectrophometry (MS).

In one embodiment, samples from the patient can be taken at regular intervals after administration of the octanol. In a particular embodiment, the samples can be collected at least every 15, 30, 45 and/or 60 minutes after administration for a certain time period, such as at least 1, 2, 3, 4, 5, 6, 7, 8, 9 or 10 hours. In a specific embodiment, the samples can be taken every 15 minutes for the first hour and then every half hour for the next two hours. In a specific embodiment, the samples can be taken every 15 minutes for the first hour and then every half hour for the next two hours and every two to four hours for 24 hours and every 6 to 8 hours for times up to 72 hours after administration.

### III. Methods of Treatment

The octanol formulations, routes of administration and dosing regimens of the present invention can be used for the treatment of tremors. In one embodiment, the treatment of essential tremor is provided. In a particular embodiment, the treatment of essential tremor with an oral formulation of octanol and soybean oil is provided. In another embodiment, the treatment of drug-induced and/or disease-induced tremor is provided via the methods and compositions described herein. In one embodiment, the drug-induced tremor can be caused by cysclosporine. In another embodiment, the drug-induced tremor can be caused by Parkinson's disease. In still further embodiments, the compositions and methods of the present invention can be used to treat a tremor selected from the group including, but not limited to physiological tremor, enhanced physiologic tremor; cerebellar tremor; Holmes' tremor; drug-induced tremor; tremor due to systemic disease such as thyrotoxicosis, hepatic failure, delirium tremens, drug withdrawal; psychogenic tremor; orthostatic tremor.

In another embodiment, the pharmaceutical formulations provided can be used to treat tremors caused by diseases or disorders including but not limited to Parkinson's disease, multiple sclerosis, stroke, head injury Wilson's disease, mercury poisoning; an over-active thyroid gland; and liver encephalopathy. In additional embodiment, the tremor to be treated can be caused by psychiatric disorders.

Additional tremor that can be treated with the compositions and methods described herein include, but are not limited to tremor associated with the following: normal aging, stress, tiredness, caffeine, alcohol, hypoglycemic attack, high blood sugar levels, anger, aggression, excitement, alcoholism, liver disease, kidney disease, stroke, hypoglycemia, brain tumors, hyperthyroidism, Friedrich's ataxia, head injury, concussion, tertiary syphilis, syphilis, seizure disorders, anxiety disorders, GAD, panic disorders, intermittent explosive disorder, alcohol withdrawal, drug withdrawal, amphetamine withdrawal, hallucinogen withdrawal, illicit drugs, cocaine, amphetamine intoxication, asthma medications, theophylline, epileptic medications, dilantin, compazine, African Sleeping sickness, autoimmune thyroid diseases, barbiturate abuse, benzodiazepine abuse, bipolar disorder, carbon monoxide poisoning, delirium tremens, dementia with lewy bodies, diabetic hypoglycemia, dystonias, Epilepsy, Fahr's Syndrome, Febrile Seizures, Friedreich's ataxia, Generalized anxiety disorder, Graves Disease, hyperthyroidism, hypoglycemia, Japanese encephalitis, Machado-Joseph Disease, Malaria, metachromatic Leukodystrophy, Multiple Sclerosis, Olivopontocerebellar atrophy, Opsoclonus Myoclonus, Panic attack, Pelizaeus-Merzbacher disease, rabies, Ramsay Runt Syndrome Type 2, Schilder's Disease, social phobia, Spinal Muscular Atrophy type III, Temporal arteritis, thyroid disorders, Toxoplasmosis and/or Wilson's Disease.

In other embodiments, the pharmaceutical formulations provided can be used to treat drug-induced tremors. In another embodiment, the pharmaceutical formulations provided can be used to treat tremor occurring as a side effect of another therapeutic agent. Tremor can occur as a side effect of drugs including neuroleptics, metoclopramide, theophylline, bronchodilators, Valproate, amiodarone, stimulants, such as cocaine and amphetamines; antidepressants, antipsychotics, caffeine, lithium, a variety of medications used to treat Parkinson's disease, asthma medications, thyroid hormone medications, and as a result of withdrawal from alcohol or addictive drugs. Types of tremors induced by drugs include enhanced physiologic tremor, rest tremor, and action tremor. Signs and symptoms of drug-induced tremors depend on the drug used and on a patient's predisposition to its side effects. Some drugs can cause extrapyramidal side effects manifesting as bradykinesia, rigidity, and tremor.

In other embodiments, the drug-induced tremor can be caused by a drug selected from but not limited to the following: cysclosporine, antidepressants, such as selective serotonin reuptake inhibitors (SSRI's) (for example, prozac, paxil); amphetamines, antipsychotics, such as haloperidol, lithium, and metoclopramide; and/or caffeine. In one embodiment, the drug-induced tremor can be caused by withdrawal from drugs such as alcohol and/or opiods.

### Combination Therapy

In further embodiments of the present invention, the octanol formulations and methods of treatment described herein can be administered in combination or alternation with another drug, such as another drug for the treatment of tremor. In one embodiment, octanol formulations, such as octanol can be used in combination and/or alternation with a drug selected from but not limited to the following: beta blockers, such as propranolol, nadolol and/or metaprolol; anticonvulsants, such as primidone, acetazolamide, methazolamide, valproic acid and/or gabapentin; benzodiazepines, such as diazepam, alprazolam, chlordiazepoxide, and/or clonazepam; and Botulinum toxin type A and/or B.

In other embodiments, the octanol formulations and methods of treatment described herein can be used in combination and/or alternation with drugs used to treat Parkinson's disease. In one embodiment, the octanol formulations described herein can be given in combination and/or alternation with a drug selected from but not limited to the following: dopaminergic agents, such as L-dopa and/or levodopa/carbidopa, dopamine agonists, such as bromocriptine, pramipexole, ropinirole and/or pergolide; COMT inhibitors, such as entacapone and/or tolcapone; monoamine oxigenase (MAO) inhibitors, such as selegiline and/or rasagiline; antiexcitatory agents, such as remacemide; anti-viral agents, such as amantadine; anticholinergics, such as trihexyphenidyl, benztropine, procyclidine, and/or biperiden; antipsychotics, such as risperidone, olanzapine, quetiapine, and/or clozapine.

In other embodiments, the octanol formulations and methods of treatment described herein can be used in combination and/or alternation with drugs used to treat multiple sclerosis. In one embodiment, the octanol formulations described herein can be given in combination and/or alternation with a drug selected from but not limited to the following: interferon beta 1 a, interferon beta 1b, glatiramer acetate, mitoxantrone, azathiprine, cyclophosphamide, cyclosporine, methotrexate, cladribine, methylprednisolone, prednisone, prednisolone, dexamethasone, acth, corticotrophin, carbamazepine, gabapentin, topiramate, zonisamide, phenyloin, desipramine, amitriptyline, imipramine, doxepin, protriptyline, cannabis, pentoxiffylline, and/or hydroxyzine.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1

### Softgel Capsule Formulation Containing 800 mg 1-Octanol

Table 1 describes a formulation for an 1-octanol in soybean oil in a softgel capsule. The formula is administered by orally ingesting the capsule. Disintegration of the capsule was measured to be less than 30 minutes. Each capsule is brown and oblong (20 minims) and should be stored away from moisture in a closed container. Expected shelf life is 2 years.

TABLE 1
Representative softgel capsule formulation (800 mg 1-Octanol)

| Name of components | Unit (mg) | Function |
|---|---|---|
| 1-octanol | 800 | Active substance |
| Soybean Oil | 200 | Diluent |
| Lemon Oil | 30 | Diluent |
| Gelatin (Bovine, certified | 300 | Capsule material |
| BSE Free) | | |
| Glycerin | 40 | Capsule material |
| Caramel | 8 | Capsule coloring agent |
| Total weight | 1378 | |

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A pharmaceutical composition comprising octanol and a pharmaceutically acceptable excipient, wherein said pharmaceutical composition is formulated for enteric release and said octanol is present in an amount sufficient to treat involuntary tremors when administered to a subject.

2. The pharmaceutical composition of claim 1, wherein said pharmaceutical composition is an enteric coated capsule.

3. The pharmaceutical composition of claim 1, wherein said pharmaceutical composition is an enteric coated tablet.

4. The pharmaceutical composition of claim 1, wherein said enteric coating comprises cellulose, polyvinyl polymers, or polyacrylic polymers.

5. The pharmaceutical composition of claim 1, wherein said octanol is 1-octanol.

6. The pharmaceutical composition of claim 1, wherein said octanol is selected from 2-octanol, 3-octanol, and 4-octanol.

7. The pharmaceutical composition of claim 1, wherein said octanol is a mixture of two or more octanols selected from 1-octanol, 2-octanol, 3-octanol, and 4-octanol.

8. A capsule comprising octanol and an oil, wherein said octanol is present in an amount sufficient to treat involuntary tremors when administered to a subject.

9. The capsule of claim 8, wherein said oil is an essential oil.

10. The capsule of claim 9, wherein said essential oil is almond-bitter oil, anise oil, anise star dark oil, gurjun balsam oil, white gurjun balsam, basil oil, bergamot oil, camphor oil, caraway oil, cassia oil, cananga oil, chamomile oil, cherry oil, cinnamon oil, citronella oil, clove stern oil, clove leaf oil, clove bud oil, cognac oil, coriander oil, cubeb oil, eucalyptus oil, eugenol oil, ginger oil, grapefruit oil, jasmine oil, laurel oil, lavender oil, lemon oil, lime oil, mace oil, mandarin oil, mayonara oil, menthol oil, mint oil, nutmeg oil, orange oil, patchouli oil, peppermint yakima oil, peppermint oil, rose oil, sage oil, sassafras oil, spearmint oil, tangerine oil, thyme oil, violet oil, vetiver oil, or wintergreen oil.

11. The capsule of claim 10, further comprising peanut oil, cottonseed oil, safflower oil, sesame oil, olive oil, corn oil, or soybean.

12. The capsule of claim 11, wherein said capsule comprises 100-300 mg of soybean oil, and 10-50 mg of lemon oil.

13. The capsule of claim 8, wherein said capsule comprises 50 mg to 1.0 g of octanol.

14. The capsule of claim 8, wherein said octanol is 1-octanol.

15. The capsule of claim 8, wherein said octanol is selected from 2-octanol, 3-octanol, and 4-octanol.

16. The capsule of claim 8, wherein said octanol is a mixture of two or more octanols selected from 1-octanol, 2-octanol, 3-octanol, and 4-octanol.

17. A pharmaceutical composition comprising an ester of an octanol and a pharmaceutically acceptable excipient, wherein said ester is present in an amount sufficient to treat involuntary tremors when administered to a subject.

18. The pharmaceutical composition of claim 17, wherein said ester is octyl acetate, octyl propionate, or octyl butyrate.

19. The pharmaceutical composition of claim 17, wherein said pharmaceutical composition is formulated for oral administration.

20. The pharmaceutical composition of claim 18, wherein said pharmaceutical composition is a capsule, tablet, syrup, or elixir.

21. A pharmaceutical composition comprising octanol and a pharmaceutically acceptable excipient, wherein said pharmaceutical composition is formulated for sustained release and wherein said octanol is present in an amount sufficient to treat involuntary tremors when administered to a subject.

22. A pharmaceutical composition comprising an ester of an octanol and a pharmaceutically acceptable excipient, wherein said pharmaceutical composition is formulated for sustained release wherein said ester is present in an amount sufficient to treat involuntary tremors when administered to a subject.

23. The pharmaceutical composition of claims 21 or 22, wherein said pharmaceutical composition is a sustained release capsule.

24. The pharmaceutical composition of claims 21 or 22, wherein said pharmaceutical composition is a sustained release tablet.

25. (canceled)

26. (canceled)

27. The pharmaceutical composition of any of claims 17, 21, or 22, wherein said octanol is selected from 1-octanol, 2-octanol, 3-octanol, 4-octanol, and mixtures thereof.

28. A method of treating an involuntary tremor in a subject in need thereof, said method comprising administering to said subject a pharmaceutical composition of any of claims 1-27 in an amount sufficient to treat said involuntary tremor.

29. The method of claim 28, wherein said pharmaceutical composition is administered two or three times daily.

30. The method of claim 28, wherein said octanol is 1-octanol.

31. The method of claim 28, wherein said octanol is selected from 2-octanol, 3-octanol, and 4-octanol.

32. The method of claim 28, wherein said octanol is a mixture of two or more octanols selected from 1-octanol, 2-octanol, 3-octanol, and 4-octanol.

33. A kit comprising (i) a pharmaceutical composition comprising an ester of an octanol and (ii) instructions for administering said pharmaceutical composition to a subject for the treatment of involuntary tremors.

34. A kit comprising (i) a pharmaceutical composition comprising octanol and formulated for enteric release and (ii) instructions for administering said pharmaceutical composition to a subject for the treatment of involuntary tremors.

35. A kit comprising (i) a pharmaceutical composition comprising octanol formulated for sustained release and (ii) instructions for administering said pharmaceutical composition to a subject for the treatment of involuntary tremors.

36. A kit comprising (i) a pharmaceutical composition comprising an ester of an octanol formulated for sustained release and (ii) instructions for administering said pharmaceutical composition to a subject for the treatment of involuntary tremors.

37. (canceled)

38. (canceled)

39. The kit of any of claims 33-36, wherein said octanol is selected from 1-octanol, 2-octanol, 3-octanol, 4-octanol, and mixtures thereof.

40. A method of treating an involuntary tremor in a subject in need thereof, said method comprising administering to said subject a pharmaceutical composition comprising two or more octanols selected from 1-octanol, 2-octanol, 3-octanol, and 4-octanol, wherein said composition is administered in an amount sufficient to treat said involuntary tremor.
